(19) United States
(12) Patent Application Publication
(10) Pub. No.: US 2008/0123913 A1
(43) Pub. Date: May 29, 2008

(54) Method and Computer Program Product For the Storage Ensuring Fast Retrieval and Efficient Transfer of Interrelated High-Volume 3D Information

(75) Inventors: Tibor Virag, Budapest (HU); Attila Laszlo, Budapest (HU); Bela Molnar, Budapest (HU); Attila Tagscherer, Tiszadorogma (HU); Viktor Sebestyen Varga, Pecel (HU)

Correspondence Address: DAVIDSON BERQUIST JACKSON & GOWDEY LLP, 4300 WILSON BLVD., 7TH FLOOR, ARLINGTON, VA, 22203, US

(73) Assignee: 3DHISTECH KFT., Budapest (HU)

(21) Appl. No.: 11/662,976

(22) PCT Filed: Jan. 27, 2005
(86) PCT No.: PCT/IB2005/050344

§ 371 Date: Nov. 7, 2007

(30) Foreign Application Priority Data
Sep. 17, 2004 (HU) P0401870

(51) Int. Cl. G06K 9/00

(52) U.S. Cl. 382/128

(57) ABSTRACT

A method for the storage ensuring fast retrieval and efficient transfer of interrelated, high volume, mainly 3D, information, whereby the input information is assigned to two files, so that image data information required for histological reconstruction is stored in one file and information regarding predetermined parameters of the image data is stored in the other file, whereas in the course of the storing of the received information, associated information regarding the hierarchical relationship of individual image data is also stored in a further file. A computer program product for the storage ensuring fast retrieval and efficient transfer of interrelated, high-volume, mainly 3D, information, contains a computer-readable storage medium including a computer-readable programme code, further a software tool to store associated information regarding the hierarchical relationship of individual image data, while storing input information.

The invention relates to a method for the storage ensuring fast retrieval and efficient transfer of interrelated, high-volume, mainly 3D, information, whereby the input information is assigned to two files, so that image data information required for histological reconstruction is stored in one file and information regarding predetermined parameters of the image data is stored in the other file. Another subject of the present invention is a computer program product containing a computer-readable storage medium including a computer-readable programme code.

Analysis of biopsy specimens is most important in medical diagnostics. Histological examinations are performed by removing a tissue sample from the body of the patient and making thin cuttings out of it. The excisions are placed on a glass plate—so-called (microscopic) slide—, and then painted and subjected to microscopic examination. It is a disadvantage of the said method that, of the 3D tissue, only a layer of a thickness of 2 to 5 μm can be examined. As for the known 3D medical diagnostic instruments, examinations by computer tomography (CT) and magnetic resonance investigation (MRI) have the disadvantage of offering a much smaller resolution capacity than the microscope, and neither can the special painting methods of histological examinations be used.

Instead of making a few cuttings out of the tissue sample, the entire sample or a significant part of it is subjected to histotomic excision. The known methods involve the fast digitalisation of all biopsy specimens and a reconstruction of the 3D image out of the cuttings. The methods known to those skilled in the art furthermore allow to view the reconstructed samples on networked workstations on the intranet or the Internet.

The useful (net) surface area of a slide is 25×50 mm. At a resolution of 0,3 μm, what could be called the standard ratio, this means approximately 83 000×166 000 pixels. The systems in use to date are not prepared to display, manipulate and transmit images of this size. If each pixel of an image of this size is represented on 3 bytes, as is the standard practice, at a compression rate of 1:10, the size of the resulting file would still be 3,8 GB.

Another problem is that doctors use object-glasses of different magnifications to examine tissue samples. The digital simulation of the use of the microscope requires the down-scaling of images made with high-resolution lenses. For example, in order to display an image made by an object-glass of 20 mm as one made by an object-glass of 4 mm, it is to be reduced in the directions x and y at a rate of five to one, that is, the entire system must handle a file that is 25 times bigger than the one actually needed. In case of Internet-based use, this may imply such deceleration as is unacceptable for the user. The digitized slide may also be used as an input for automatic measurement algorithms. Such algorithms can perform a series of measurements that are hardly or not at all feasible by traditional methods. The relevant measurement results, however, must also be displayed on the screen, but this raises the same problems as displaying the images themselves. Even if the system displays a small section only of the slide, it must still manage the entire data set. An alternative solution is for the system to select the measurement data associated with the given segment, but this is a slow process requiring high-capacity machines.

Most image formats store a maximum of three colour channels. The known microscopes, on the other hand, allow the fitting of fluorescent filters over the lens, and one sample can be digitized at the corresponding number of wavelengths, which increases the resulting data quantity even further.

U.S. Pat. No. 6,272,235 describes a method and equipment for the purpose of creating a virtual microscope slide. The method consists of the digitalisation of the slide, that is, its reading into the memory and, subsequently, the arrangement of the input frames so that said frames can be viewed, firstly, conveniently, even without microscope, and, secondly, they can be transferred easily to one or several remote locations, to be viewed there. The method consists of making several original microscope images of the slide, digitizing them at small magnification, and then storing said digitized frames so that they be inter-linked in a standardised way and hence produce a full-scale virtual image of the slide read at small magnification. Furthermore, several original microscope images are made at higher magnifications, too, which are then digitized and stored again so that the digitized images make up a single, continuous, coherent image and hence show the high-resolution virtual macro image of the slide. In connection with these virtual, digitized, macro and micro images, the method also creates a data structure containing the mapping co-ordinates of the individual images as well. In order to be able to view the images that have been read, digitized, stored and occasionally also compiled, the solution also proposes a general browsing/viewing programme in the data structure allowing remote users to display the images compiled of the frames and to manipulate, if need be, the image displayed on their own, remote, screen. In order to ensure the reasonable use of the imaging and data structure outlined above away from their place of origin, by transfer through data transmission and communication channels, often characterised by low band-widths, to remote locations, the data quantity must inevitably be compressed to a large extent which is a “lossy” process in most cases, although such data loss is contrary to the demand that the transferred images be suitable for detailed analysis at remote locations if needed. The interactive general browser developed by this solution is partly meant to fulfil this task, allowing as it does the person viewing the frames to navigate up and down, left and right, and look at those areas which are of interest to him/her. One typical feature of the browser is the marker provided for the end-user in the macro image, telling the exact location of the actual micro image, and helping the user select the areas of interest to him/her from the macro image and then subjecting them to more detailed examination through higher-resolution digitized images.

Despite the advantage of reducing the storage capacity demand outlined in the introduction, this solution has the deficiency that navigation in the images created this way is still time-consuming, a phenomenon manifesting itself not only at remote sites, but occasionally even at the server used to store the data concerned.

Therefore, an objective of the present invention is to work out a method whereby, while retaining the system- and user-specific advantages of the solutions known so far, it is possible to ensure fast and reliable navigation in a very large database, whether it is used on-site or remotely, e.g. through the Internet, so that data storage, retrieval and display should not require the costly upgrading of the available instrument pool or the deployment of new equipment, a costly move in any case.

The present invention is based on the recognition that, in addition to the data sets known so far, another, auxiliary, data set must be compiled, exclusively for the purpose of promoting the tracking and identification of data segments selected stochastically, at random, and offering users a search facility in a transparent and easily manageable form.

The preferred embodiments of the proposed method are described under the Claims below.

Our proposed solution to the objective set above is based on a method for the storage ensuring fast retrieval and efficient transfer of interrelated, high-volume, mainly 3D, information, whereby the input information is assigned to two files, so that image data information required for histological reconstruction is stored in one file, and information regarding predetermined parameters of the image data is stored in the other file, whereas in the course of the storing of the received information, associated information regarding the hierarchical relationship of individual image data is also stored in a further file.

According to one advantageous implementation of the proposed method slides digitized as information are being used.

It is also advantageous according to the present proposal to store the files of each digitized slide are in a storage unit linked to the digitized slide.

It is furthermore advantageous according to the present proposal that a directory is used in each case as storage unit.

According to another advantageous implementation of the proposed method the digitized slides are being stored in separate storage units.

According to another advantageous implementation of the proposed method a directory is used in each case as storage unit.

It is furthermore advantageous according to the present proposal that information on versions is also stored in the files.

Our proposed solution to the objective set above is based further on a computer program product for the storage ensuring fast retrieval and efficient transfer of interrelated, high-volume, mainly 3D, information, whereby the input information is assigned to two files, so that image data information required for histological reconstruction is stored in one file, and information regarding predetermined parameters of the image data is stored in the other file, containing a computer-readable storage medium including a computer-readable programme code, further a software tool to store associated information regarding the hierarchical relationship of individual image data, while storing input information.

In the following, the proposed solution will be described in more detail with reference to the attached drawings showing an exemplary embodiment of the data structure created by the proposed method, whereas

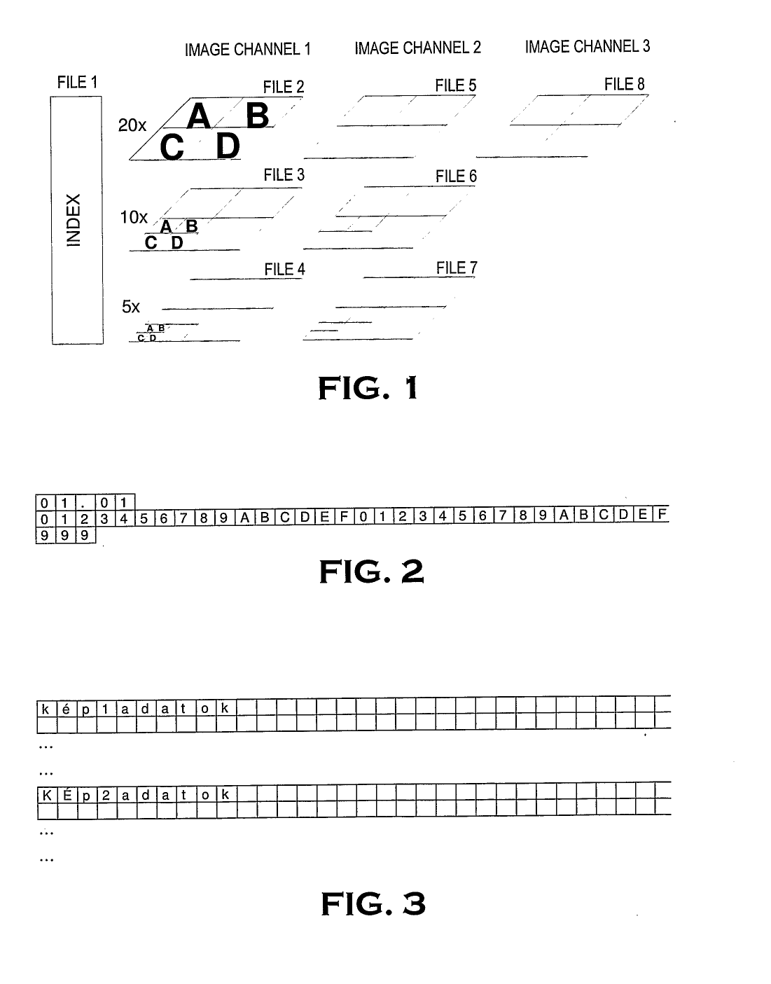

FIG. 1 shows a possible embodiment of the files making up the digital slide and their assignment according to the present invention,

FIG. 2 shows a possible structure of the header of a data file containing the image data of the frames making up the digitized slide,

FIG. 3 shows a possible arrangement of the image data of the data file filling the available space compactly,

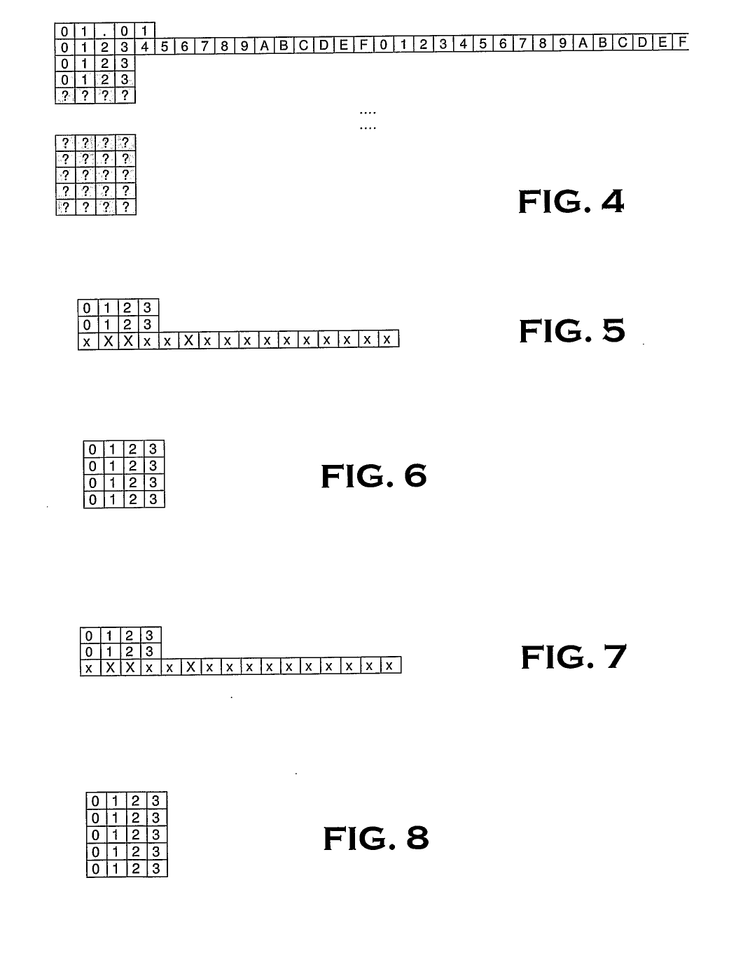

FIG. 4 shows a possible structure of the header of the table file,

FIG. 5 shows a possible embodiment of the data block representing the layer-specific table data in the table of hierarchical layers,

FIG. 6 shows the structure of a complete record of layer-specific table recordings in the table of hierarchical layers,

FIG. 7 shows a possible embodiment of the data block of the table block making up the layer-specific table data in the table of non-hierarchical layers, and

FIG. 8 shows the structure of a complete record of layer-specific table recordings in the table of non-hierarchical layers.

A data storage format developed specially for this task is used to solve the relevant problems.

The term “channel” means either a fluorescent channel, defined by the fluorescent filter block used to record the channel, or by the traditional trough-light illumination.

Given the fact that each digitized slide is associated with several files, each digitized slide is stored in a separate directory of the storage system.

The principle of storage is as follows. One file will always contain the fields of view associated with a given channel and magnification. To speed up display, one field of view is reduced to several fields of smaller magnification already in the digitalisation phase, by always halving the given image in the directions X and Y belonging to coordinates. For example, if an image of 1024×768 pixels is recorded by 20-fold magnification of a given field of view, the reduced images consisting of 512×384, 256×192, 128×96, 32×24, 16×12 and 4×3 pixels, respectively, will be generated as well. Images smaller than that would already result in pixel expressed in fractions, and, anyway, this minimum magnification is sufficient from the point of view of the application, too. The images in question are stored in separate files. One file includes not only one image, but as many of the images of the given size belonging to the given channel as possible. Every file has a pre-defined upper limit. If no more images fit into the file, a new file is opened and the remaining images are written into that. It is expedient to define the upper limit of the files so that they can be adapted to a data carrier of some kind. If, for example, digitized slides are to be archived on CD, and one digitized slide is bigger than the storage capacity of a CD, no problem will be encountered if the maximum limit is set at the maximum storage capacity of the CD. Certain hard-disc-based file systems also have upper file size limits, hence it will be easy to match this solution to these, too.

Faster display is again the main reason why images of different magnification are assigned to separate files. Generally, at a given moment, the end-user will view the slide on the screen using a single magnification only. Thanks to the sequential recording of the fields of view, images associated with juxtaposed fields of view will be sequential in the file, too, allowing optimum management of the files and their display. If images of all magnifications associated with a given channel were stored in one file, then, for example, to display a small magnification, instead of sequential reading, after each field of view, the operating system and a hard disk serving as storage medium would have to find in the file the next field of view. From another aspect, given the fact that the size of the fields of view diminishes exponentially, the use of an infinitesimal part of the file would require moving about in the entire file.

A natural approach adopted in similar systems is to store one image in one file, as in the case of digital photo cameras. This method has the drawback that, smaller magnifications included, ten thousands of files are needed for storing a single slide, and the management of these is not optimal from any point of view.

The images can be stored in the file in any format whatsoever. In the current preferred embodiment of the method, JPEG and JPEG2000 formats can be used for the purpose of lossy compression, and TIFF, BMP, PNG or JPEG2000 formats for the purpose of lossless storage.

Current IT systems can manage images of several gigabytes hardly or not at all, and neither is their operation optimal in the case of very small images. For example, the header of the commonly used image formats describing the features of the image in the file is usually much bigger than the image information content of an image of 4×3 pixels. For the image to be displayed on the screen, the relevant application must issue a system call. While the operating system can display an image of 1024×768 pixels in one step, in order to fill the same image area by images of 3×4 pixels each, the image display function must be called 65 536 times, which makes the process much slower. Therefore, in the course of digitalisation, the image of an optical field is not only reduced, but several small images are also joined into one image. To stick to the previous example: the original image of 1024×768 pixels is treated as one image, while for smaller magnifications, four of the 512×384 pixel ones are linked and treated as one image, and so on.

The slide format stores the graphic outcomes of automatic evaluations in the form of a new channel in every respect. Graphic outcome may mean, for example, that the application automatically identifies cells and glands in the tissue sample, and creates a mask for these. A gland mask, for example, will cover the given gland completely in the image.

Numeric evaluation data are also stored by field of view. If the evaluation application, for example, measured the surface of every gland in square micrometer, this data set will be distributed so that data associated with one field of view form one group, and this data group will be entered in the file as one unit, as if it were one image. As reduction cannot be interpreted for numeric data in the same way as for images, channels containing such data will consist of a single layer.

As compressed images may differ in size and hence it is impossible to calculate in advance where exactly an image associated with a given view-field will be located in the file, to ensure fast image access, an index file is created, specifying for an image of a given magnification in a given channel of a given view-field the byte it starts from within the file and its length in bytes.

FIG. 1 shows an example of a preferred embodiment according to the present invention of the files making up the digital slide. For the sake of easy access, digital slides belonging together are stored in a separate unit, for example, a directory of a storage unit and the files of the digital slides are also stored in separate directories.

In what follows, we shall describe a preferred embodiment of the files storing the digitized slide data created by the method according to the present invention. The method creates three types of files:

1) a central description file designed to include all information associated with the digitized slide or links pointing to the files including them;

2) a table file describing the hierarchy of the frames making up the digitized slide; and

3) a data file containing the image data of the frames making up the digitized slide.

In what follows, we shall describe exemplary structures of the above three file types in more detail.

Structure of a Data File

1) The inner structure of the header of the data file outlined in FIG. 2 is the following:
File version number: CHAR(5)=structured as ‘nn.nn’
Individual identifier of the digitized slide: CHAR(32)=‘hhhh . . . hhh’ (32 hexa digits): a 128-byte hexadecimal random number, to be produced by the in-built RND (random) function in chunks of 16 bytes.
Sequential number of the file within the slide: CHAR(3)=‘nnn’. It shows the sequential number of the given file within a digitized slide, its possible values being 0 . . . 239, that is, the number of files that can make up 1 digitized slide. The numeric value is stored in the form of a character string, decimally.

2) The image data of the data file outlined in FIG. 3 will fill the available space compactly. The starting point of each image is indicated in the pointer table, which is to be found in the table file. Information required for interpreting the image data (coding, file format etc.) is to be found in the central description file.

Structure of a Table File

1) The inner structure of the header of the table file outlined in FIG. 4 is as follows:
File version number: CHAR(5)=structured as ‘nn.nn’
Individual identifier of the digitized slide: CHAR(32)=‘hhhh . . . hhh’ (32 hexa digits): a 128-byte hexadecimal random number, to be produced by the in-built RND (random) function in chunks of 16 bytes.
Starting address of the table of hierarchical (tree-structured) layers within this file (INT32)
Starting address of the table of non-hierarchical layers within this file (INT32)
Place reserved for another 62 pieces of INT32 recordings (248 bytes)

2) Table of hierarchical (tree-structured) layers:

The table includes the pointers to the images in a two-level hierarchy. At the upper level, there is a multi-dimensional table (hierarchy table) including pointers pointing to the two-dimension tables (layer-level table) at the lower level.
a) Hierarchy table: this is a multi-dimensional table describing the interrelationships of different hierarchically interrelated digitized slides and/or levels within these, and defining the starting points of tables including layer-specific data. The hierarchy table is an array of dim1*dim2* . . . *dimN, the elements of which are 4-byte binary values each, defining the starting point of the table describing the given layer level relative to the beginning of the file. The addressing of the hierarchy table will be described later on.
b) Layer-specific table data: this is not a traditional array of n*m but a linked list consisting of table blocks. The structure of one block in the table is as follows:
   Number of valuable data records in this data block (INT32);
   Address of the subsequent block, that is, offset relative to the beginning of the file (INT32) (if there is no subsequent block in the list, its value is 0);
Data block: fixed size data area (that is, its size is independent of the value of the “number of data records”), the size of which is indicated in the PAGELENGTH field of the central description file, corresponding to the size of the data record*PAGEELEMENTCOUNT (standard value: e.g. 2048 byte usually), see FIG. 5.
c) The structure of layer-specific table recordings is provided by the following record structure:
   A value calculated from the relationship X+Y*W (where Y and Y are the co-ordinates of the frame within the slide, W is the width of the digitized slide in frames) (INT32);
   Beginning of image datum relative to the beginning of the given data file, being a 4-byte binary value (INT32);
   Length of image data measured in bytes, being a 4-byte binary value (INT32);
   Index of the data file containing the frame (INT32).

Hence in the example shown here, the size of the entire record shown in FIG. 6 is 16 byte.

3) Table of non-hierarchical layers:

The table includes the pointers to the images in a two-level hierarchy. At the upper level, there is an array (hierarchy table) including pointers pointing to the arrays (layer-level tables) at the lower level.
a) First-level table: this is a table including the starting points of tables describing various non-hierarchically related digitized slide layers. The first-level table is an array of dim1+dim2+ . . . +dimN, the elements of which are 4-byte binary values each, defining the starting point of the table describing the given layer level relative to the beginning of the file.
b) Layer-specific table data: this is not a traditional n*m array but a linked list consisting of table blocks. The structure of one block in the table is as follows:
   Number of valuable data records in this data block (INT32);
   Address of the subsequent block, that is, offset relative to the beginning of the file (INT32) (if there is no subsequent block in the list, its value is 0);
   Data block: fixed size data area (that is, its size is independent of the value of the “number of data records”), the size of which is indicated in the PAGELENGTH field of the central description file, corresponding to (the size of the data record*PAGEELEMENTCOUNT (standard value: e.g. 2048 byte usually), see FIG. 7.
c) The structure of layer-specific table recordings is provided by the following record structure:
   X: co-ordinate of the frame within the digitized slide (INT32);
   Y: co-ordinate of the frame within the digitized slide (INT32);
   Beginning of image datum relative to the beginning of the given data file, being a 4-byte binary value (INT32);
   Length of image data measured in bytes, being a 4-byte binary value (INT32);
   Index of the data file containing the frame (INT32). Hence in the example shown here, the size of the entire record shown in FIG. 8 is 20 byte.

Central Description File:

The function of this file is to store all sorts of information relating to the digitized slide. The file often includes links only to other files which actually store the data. The general rule in this respect is that this file contains exclusively a description of the physical properties of the digitized slide, whereas image data and any related other data (e.g. data of the patient whose digitized slide it is etc.) are only recorded in the file in the form of links.

As for the structure of the file, it is an INI file. Its structure includes the following sections:

1) General physical information (GENERAL section): the majority of the physical data obtained from the digitized slide is to be found here, including the following:
SLIDE_ID: a 128-byte random number generated by the relevant application at the time of the creation of the digitized slide. The numeric value is stored in the file as a 32-digit hexadecimal number, and serves as individual identifier of the digitized slide. The given random number is generated in chunks of 16-byte by the in-built RND function of the development environment.
IMAGE_OVERLAP_MICROMETERS_X,
   IMAGE_OVERLAP_MICROMETERS_Y: the value of the overlap between the frames making up the digitized slide in horizontal and vertical direction, respectively, expressed in micrometers.
SLIDE_POSITION_X, SLIDE_POSITION_Y: position of the starting point of the digitized slide expressed in micrometer. If the digitized biopsy cutting has a special calibration point, then this starting point co-ordinate is measured relative to the given calibration point as origin. If the cutting has no such point, the point is given relative to the absolute zero point of the stage.
SLIDE_WIDTH, SLIDE_HEIGHT: size, that is, width and height, of the digitized slide, expressed in micrometer.
SLIDE_MARKER_X, SLIDE_MARKER_Y: position of the calibration point of the digitized slide within the absolute co-ordinate system of the stage, in micrometer.
IMAGE_FILE_FORMAT, COMPRESSED, COMPRESSION: the parameters defining the method and data format of the compression of the frames making up the digitized slide.
PAINT_TYPE, SLIDE_TYPE, SLIDE_COMMENT: these include other features of the digitized slide (e.g., type of painting of the biopsy cutting, type of the digitized slide, user's comments etc.).
PHYSICAL_MAGNIFY: physical magnification value of the microscope.

IMAGENUMBER_X, IMAGENUMBER_Y: expresses the extension of the levels measured in frames at each level of the digitized slide (this extension refers exclusively to the hierarchical layers—non-hierarchical layers have individually definable (and alterable) sizes.
PROJECT_NAME, SLIDE_NAME: name of the project including the digitized slide and/or name of the digitized slide.
SLIDE_VERSION: database format version of the digitized slide.

2) Table file hierarchy information (HIERARCHICAL): this section contains the description of the hierarchical structure of the digitized slide.
INDEXFILE: parameter including the name of the table file.
HIER_COUNT: number of the hierarchical levels of the digitized slide.
NONHIER_COUNT: number of the non-hierarchical layers of the digitized slide.
PAGEELEMENTCOUNT: maximum number of elements occurring in the data block in the file. Its explanation is to be found in the description of the table file.
PAGELENGTH: aggregate size of the maximum number of elements occurring in the data block in the table file (not including the header of the data block). Its explanation is to be found in the description of the table file.
HIER_{—}0_NAME: name of the physical parameter constituting the lowest-rank hierarchy level.
HIER_{—}0_COUNT: number of special values of the parameter constituting the lowest-rank hierarchy level.
HIER_{—}0_SECTION: name of section of supplementary values associated with the parameter constituting the lowest-rank hierarchy level.
HIER_{—}0_DEFAULT: sequence number of the default value of the parameter constituting the lowest-rank hierarchy level (if none, its value is 1).
HIER_{—}0_VAL_{—}0: 1^{st} possible value of the parameter constituting the lowest-rank hierarchy level.
HIER_{—}0_VAL_{—}0_SECTION: name of section in the file including other parameters associated with the 1^{st} possible value of the parameter constituting the lowest-rank hierarchy level.
HIER_{—}0_VAL_{—}1: 2^{nd} possible value of the parameter constituting the lowest-rank hierarchy level.
HIER_{—}0_VAL_{—}1_SECTION: name of section in the file including other parameters associated with the 2^{nd} possible value of the parameter constituting the lowest-rank hierarchy level.
HIER_{—}0_VAL_{—}1_SECTION: name of section in the file including other parameters associated with the 2^{nd} possible value of the parameter constituting the lowest-rank hierarchy level.
. . .
HIER_{—}0_VAL_N−1: n^{th} possible value of the parameter constituting the lowest-rank hierarchy level.
HIER_{—}0_VAL_N−1_SECTION: name of section in the file including other parameters associated with the n^{th} possible value of the parameter constituting the lowest-rank hierarchy level.
. . .
. . .
HIER_M−1_NAME: name of the physical parameter constituting the highest-rank hierarchy level.
HIER_M−1_COUNT: number of special values of the parameter constituting the highest-rank hierarchy level (M^{th} level)
HIER_M−1_DEFAULT: sequence number of the default value of the parameter constituting the highest-rank hierarchy level (if none, its value is 1).
HIER_M−1_SECTION: name of section of supplementary values associated with the parameter constituting the highest-rank hierarchy level in the file.
HIER_M−1_VAL_{—}0: 1. possible value of the parameter constituting the highest-rank hierarchy level (M^{th} level).
HIER_M−1_VAL_{—}0_SECTION: name of section in the file including other parameters associated with the 1^{st} possible value of the parameter constituting the highest-rank hierarchy level.
HIER_M−1_VAL_{—}1: 2^{nd} possible value of the parameter constituting the highest-rank hierarchy level (M^{th} level).
HIER_M−1_VAL_{—}1_SECTION: name of section in the file including other parameters associated with the 2^{nd} possible value of the parameter constituting the highest-rank hierarchy level
. . .
HIER_M−1_VAL_−1: k. possible value of the parameter constituting the highest-rank hierarchy level (M^{th} level).
HIER_M−1_VAL_K−1_SECTION: name of section in the file including other parameters associated with the k. possible value of the parameter constituting the highest-rank hierarchy level.
NONHIER_{—}0_NAME: name of the very first non-hierarchical layer.
NONHIER_{—}0_COUNT: number of levels in the very first non-hierarchical layer.
NONHIER_{—}0_SECTION: name of section in this file of supplementary values in the very first non-hierarchical layer.
NONHIER_{—}0_VAL_{—}0: name of first level in the very first non-hierarchical layer.
NONHIER_{—}0_VAL_{—}0_SECTION: name of section in this file of other parameters related to the first level in the very first non-hierarchical layer.
NONHIER_{—}0_VAL_{—}0_IMAGENUMBER_X: X extension of the first level in the very first non-hierarchical layer.
NONHIER_{—}0_VAL_{—}0_IMAGENUMBER_Y: Y extension of the first level in the very first non-hierarchical layer.
. . .
NONHIER_{—}0_VAL_N−1: name of N^{th} level in the very first non-hierarchical layer.
NONHIER_{—}0_VAL_N−1_SECTION: name of section in this file of other parameters related to the N^{th} level in the very first non-hierarchical layer.
NONHIER_{—}0_VAL_N−1_IMAGENUMBER_X: X extension of the N^{th} level in the very first non-hierarchical layer.
NONHIER_{—}0_VAL_N−1_IMAGENUMBER_Y: Y extension of the N^{th} level in the very first non-hierarchical layer.
. . .
. . .
NONHIER_M_NAME: name of the M^{th} non-hierarchical layer.
NONHIER_M_COUNT: number of levels in the M^{th} non-hierarchical layer
NONHIER_M_SECTION: name of section in this file of supplementary values in the M^{th} non-hierarchical layer.
NONHIER_M_VAL_{—}0: name of first level in the M^{th} non-hierarchical layer.
NONHIER_M_VAL_{—}0_SECTION: name of section in the file including other parameters associated with the first level in the M^{th} non-hierarchical layer.
NONHIER_M_VAL_{—}0_IMAGENUMBER_X: X extension of the first level in the M^{th} non-hierarchical layer.
NONHIER_M_VAL_{—}0_IMAGENUMBER_Y: Y extension of the first level in the M^{th} non-hierarchical layer.
. . .
NONHIER_M_VAL_K−1: name of K^{th} level in the M^{th} non-hierarchical layer.
NONHIER_M_VAL_K−1_SECTION: name of section in the file including other parameters associated with the K^{th} first level in the M^{th} non-hierarchical layer.
NONHIER_M_VAL_K−1_IMAGENUMBER_X: X extension of the K^{th} level in the M^{th} non-hierarchical layer.
NONHIER_{—}0_VAL_K−1_IMAGENUMBER_Y: Y extension of the K^{th} level in the M^{th} non-hierarchical layer.

Characteristic features of layers described by HIER . . . parameters:

This structures include the element numbers of the dimensions of an array as well as the name of the physical parameters stored in the said dimensions in the layers. Currently, the following physical parameters are conceivable:
Microscope illumination value: (‘LAMP’). Possible values: the values to be set/read on the microscope lamp controlling hardware, expressed in numbers. In practice, usually, a digitized slide is recorded with a single lamp setting value, hence the extension of the dimension of this hierarchy level is 1. That is, in terms of hierarchy, this dimension can be omitted, but it is necessary to include it in the list because of the value of the parameter.
Camera integration time (‘CAMERA_TIME’). Possible values: the values to be set/read on the camera hardware, expressed in half-frame (field) duration. In practice, usually, a digitized slide is recorded with a single integration time, hence the extension of the dimension of this hierarchy level is 1. That is, in terms of hierarchy, this dimension can be omitted, but it is necessary to list it because of the value of the parameter.
Microscope filter setting (‘SLIDE FILTER LEVEL’). Possible values: the positions of the filter to be set/read on the microscope hardware, occasionally together with the name of the filter. Usually, a digitized slide is recorded with one type of filter only, but for fluorescent images, for example, this value may be higher.
Microscope focus plane (‘MICROSCOPE FOCUS LEVEL’). Possible values: the focal length values expressed relative to the sharp plane, that is, with +/− sign, in nm. In practice, the element number of this dimension is often higher than 1.
Magnification level (‘SLIDE ZOOM LEVEL’). Possible values: the ratios provided relative to the magnification of the original image (the one shown by the microscope). In practice, usually 3 levels occur: 1, ½, ¼.
Further parameters, which, in principle, can be altered mid-way, during the digitization of the slide, from frame to frame.

At the given parameter levels, each parameter value may be associated with a separate section including the parameters relating to the given parameter values of the given digitized slide level. A good example is the parameter SLIDE ZOOM LEVEL: every one of its possible values is associated with a set of values of the following physical features:
MICROMETER_PER_PIXEL_X, MICROMETER_PER_PIXEL_Y: its value is the pixel size at the given ZOOM level
DIGITIZER_WIDTH, DIGITIZER_HEIGHT: its value is the image size at the given ZOOM level, measured in pixels.

Characteristic features of layers described by NONHIER . . . parameters:

The layers described this way are fully independent of one another; each layer defines the number and name of levels to be found in it. The parameters of the individual levels, on the other hand, specify the extension of the levels (in the directions X and Y). Currently, the following non-hierarchical layers are conceivable:
Description of 3D objects
Headers of various XML layers
Further parameters, which are not associated with frames, but with the entire digitized slide.

At the given parameter levels, each parameter value may be associated with a separate section including the parameters relating to the given parameter values of the given digitized slide level. The extension of the dimensions of the hierarchy tables is defined in the table file by the hierarchy and, within that, the extension of the dimensions, recorded in this section.

3) Data file information (‘DATAFILE’): this section includes the number and name of data files containing image data:
FILE_COUNT: parameter defining the number of data files (in the present example, a maximum of 240 data files is allowed),
FILE_{—}0, FILE_{—}1, . . . , FILE_N: parameters storing the name of the N+1 data file

4) Link information (‘LINKS’): this section includes links to attached information not to be detailed here:
LINK_COUNT: defines the number of links.
LINK_{—}1_NAME: name of the 1^{st} link.
LINK_{—}1_FILE: name of file containing the linked data.
. . .
LINK_N_NAME: name of the n^{th} link.
LINK_N_FILE: name of file containing the linked data.

5) Further sections, and parameters within them, the name of which is defined by the records provided in the HIERARCHICAL section, to be described in more detail there.

The following table shows an exemplary embodiment of the addressing of the hierarchy table of the hierarchical layer levels.

| | | | | | |
|---|---|---|---|---|---|
| Hierarchy level (index) in the central | H_HIER_COUNT | . . . | H2 | H1 | H0 |
| description file (INI file) | | | | | |
| HIER_COUNT indicates its number | | | | | |
| Number of elements within the hierarchy | n_HIER_COUNT | . . . | n2 | n1 | n0 |
| (number of different elements that are | | | | | |
| possible within one hierarchy plane) (radix) | | | | | |
| HIER_i_COUNT | | | | | |
| Initial value of the given hierarchy plane | d_HIER_COUNT | . . . | d2 | d1 | d0 |
| (if the given hierarchy plane is not known in | | | | | |
| the processing unit, then the hierarchy plane | | | | | |
| must be addressed there with the initial | | | | | |
| value) HIER_i_DEFAULT | | | | | |
| Index used to address hierarchy level | idx_HIER_COUNT | . . . | idx2 | idx1 | idx0 |

The dec numeric value can be used to address the hierarchy table of the table file:

dec=idx0+n0*idx1+n0*idx2+ . . . +n0*n1*n2* . . . *n_HIER_COUNT*idx_HIER_COUNT

If idx_i is not used, then d_i is used instead.

The following table on the next page shows a specific exemplary embodiment of the central description file modelled on the structure outlined above:

[GENERAL]
DIGITALIZED SLIDE_VERSION = 01.02
DIGITALIZED SLIDE_ID = 0123456789ABCDEF0123456789ABCDEF
IMAGE_OVERLAP_MICROMETERS_X = 10.12
IMAGE_OVERLAP_MICROMETERS_Y = 12.13
DIGITALIZED SLIDE_POSITION_X = 9234.12
DIGITALIZED SLIDE_POSITION_Y = 8245.13
DIGITALIZED SLIDE_WIDTH = 70234.12
DIGITALIZED SLIDE_HEIGHT = 20234.13
DIGITALIZED SLIDE_MARKER_X = 5000.12
DIGITALIZED SLIDE_MARKER_Y = 2345.13
IMAGE_FILE_FORMAT = JPEG
COMPRESSED = YES
COMPRESSION = LOSSY
PAINT_TYPE = DIGITALIZED SLIDE paint type
DIGITALIZED SLIDE_TYPE = DIGITALIZED SLIDE type
DIGITALIZED SLIDE_COMMENT = comment for
DIGITALIZED SLIDE
PHISICAL_MAGNIFY = 200.0
IMAGENUMBER_X = 458
IMAGENUMBER_Y = 98
PROJECT_NAME = Konv
DIGITALIZED SLIDE_NAME=Mb1
[HIERARCHICAL]
INDEXFILE = index.dat
HIER_COUNT = 5 ; number of the hierarchical layers
of the digitalised slide
NONHIER_COUNT = 2 ; number of the non-hierarchical
layers of the digitalised slide
PAGEELEMENTCOUNT=128
PAGELENGTH=2048
HIER_0_NAME = DIGITALIZED SLIDE zoom level
HIER_0_COUNT = 3
HIER_0_DEFAULT = 1
HIER_0_VAL_0 = Normal
HIER_0_VAL_0_SECTION =
HIER_0_VAL_1 = Half
HIER_0_VAL_1_SECTION =
HIER_0_VAL_2 = Quarter
HIER_0_VAL_2_SECTION =
HIER_1_NAME = Microscope lamp level
HIER_1_COUNT = 1
HIER_1_DEFAULT = 1
HIER_1_VAL_0 = 3.42
HIER_1_VAL_0_SECTION = LAMP01
HIER_2_NAME = Camera integration time
HIER_2_COUNT = 2
HIER_2_DEFAULT = 2
HIER_2_VAL_0 = 10 ms
HIER_2_VAL_0_SECTION = CAM01
HIER_2_VAL_1 = 50 ms
HIER_2_VAL_1_SECTION = CAM02
HIER_3_NAME = Microscope filter
HIER_3_COUNT = 3
HIER_3_DEFAULT = 1
HIER_3_VAL_0 = filter 1
HIER_3_VAL_0_SECTION =
HIER_3_VAL_1 = filter 2
HIER_3_VAL_1_SECTION =
HIER_3_VAL_2 = filter 3
HIER_3_VAL_2_SECTION =
HIER_4_NAME = Microscope focus level
HIER_4_COUNT = 3
HIER_4_DEFAULT = 2
HIER_4_VAL_0 = plus 150 nm
HIER_4_VAL_0_SECTION = ZOOM01
HIER_4_VAL_1 = focus szint
HIER_4_VAL_1_SECTION = ZOOM02
HIER_4_VAL_2 = minus 150 nm
HIER_4_VAL_2_SECTION = ZOOM03
NONHIER_0_NAME = 3DData
NONHIER_0_COUNT = 1
NONHIER_0_VAL_0 = 3DObjectData
NONHIER_0_VAL_0_SECTION =
NONHIER_0_VAL_0_IMAGENUMBER_X = 2
NONHIER_0_VAL_0_IMAGENUMBER_Y = 1
NONHIER_1_NAME = XMLHeader
NONHIER_1_COUNT = 2
NONHIER_1_VAL_0 = CellParamsHeader
NONHIER_1_VAL_0_SECTION =
NONHIER_1_VAL_0_IMAGENUMBER_X = 1
NONHIER_1_VAL_0_IMAGENUMBER_Y = 1
NONHIER_1_VAL_1 = MirigyParamsHeader
NONHIER_1_VAL_1_SECTION =
NONHIER_1_VAL_1_IMAGENUMBER_X = 1
NONHIER_1_VAL_1_IMAGENUMBER_Y = 1
NONHIER_1_VAL_2 = HamParamsHeader
NONHIER_1_VAL_2_SECTION =
NONHIER_1_VAL_2_IMAGENUMBER_X = 1
NONHIER_1_VAL_2_IMAGENUMBER_Y = 1
[DATAFILE]
FILE_COUNT = 2
FILE_1 = dat01.dat
FILE_2 = dat02.dat
[LINKS]
LINK_COUNT = 2
LINK_1_NAME = Program 1 settings
LINK_1_FILE = file1.ini
LINK_2_NAME = Program 2 settings
LINK_2_FILE = file2.ini
; other sections (depending on earlier variables):
[CAM01]
CAMERA_OTHER_SETTING = Other setting 1
[CAM02]
CAMERA_OTHER_SETTING = Other setting 2
[ZOOM01]
MICROMETER_PER_PIXEL_X = 0.123456
MICROMETER_PER_PIXEL_Y = 0.135458
DIGITIZER_WIDTH = 768
DIGITIZER_HEIGHT = 576
[ZOOM02]
MICROMETER_PER_PIXEL_X = 0.246912
MICROMETER_PER_PIXEL_Y = 0.270916
DIGITIZER_WIDTH = 384
DIGITIZER_HEIGHT = 288
[ZOOM03]
MICROMETER_PER_PIXEL_X = 0.493824
MICROMETER_PER_PIXEL_Y = 0.541832
DIGITIZER_WIDTH = 192
DIGITIZER_HEIGHT = 144
[LAMP01]
LAMP_OTHER_SETTINGS = Other lamp setting 1

1-8. (canceled)

9. A method for ensuring the storage, fast retrieval and efficient transfer of interrelated, high-volume 3D information, whereby the input information is assigned to two files, so that image data information required for histological reconstruction is stored in one of the two files, and information regarding predetermined parameters of the image data is stored in the other of the two files, wherein during the step of storing received information, associated information regarding the hierarchical relationship of individual image data is stored in a further file.

10. The method according to claim 9, including the step of using digitized slides as the input information.

11. The method according to claim 10 further including the step storing the files of each digitized slide in a storage unit linked to the digitized slide.

12. The method according to claim 11, further including using a directory in each case as storage unit.

13. The method according to claim 12, wherein the digitized slides are stored in separate storage units.

14. The method according to claim 13, wherein a directory is used in each case as storage unit.

15. The method according to claim 14, wherein there are a plurality of versions of slides and information on each such version of a slide is also stored in files.

16. A computer program product for ensuring the storage, fast retrieval and efficient transfer of interrelated, high-volume, mainly 3-D, information, whereby input information is assigned to two files, the input information including image data information required for histological reconstruction that is stored in one file, and information regarding predetermined parameters of the image data is stored in the other file, the computer program product further including a computer-readable storage medium including a computer-readable program code, wherein the computer program product has a software tool permitting the storage of associated information regarding the hierarchical relationship of individual image while storing the input information.
